(19) United States
(12) Patent Application Publication
(10) Pub. No.: US 2019/0192177 A1
(43) Pub. Date: Jun. 27, 2019

(54) REUSABLE NAVIGATION GUIDEWIRE

(71) Applicant: Acclarent, Inc., Irvine, CA (US)

(72) Inventors: Jetmir Palushi, Irvine, CA (US); Itzhak Fang, Irvine, CA (US); James Patrick Garvey, II, Mountain View, CA (US)

(21) Appl. No.: 15/852,530

(22) Filed: Dec. 22, 2017

(51) Int. Cl. A61B 17/24; A61B 34/20; A61M 25/00; A61M 25/09; A61M 25/01

(52) U.S. Cl. CPC A61B 17/24; A61B 34/20; A61M 25/0041; A61B 5/062; A61M 25/0097; A61M 25/0105; A61M 25/09

(57) ABSTRACT

A guidewire includes an elongate tube, a connector, a sensor, and a conduit. The elongate tube includes a proximal portion and a distal portion. The elongate tube has a tubular profile with a substantially smooth surface extending between the proximal portion and the distal portion. The distal portion has an outer diameter sized to fit within a drainage passageway of a paranasal sinus. The elongate tube is formed of a material configured to tolerate sterilization. The connector is configured to communicate with a navigation system. The elongate tube is rotatable relative to the connector. The conduit extends from the connector to the sensor. The conduit is configured to communicate a signal from the sensor to the connector.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically.

While several systems and methods have been made and used in ENT procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument assembly, with a guidewire in a proximal position, and with a dilation catheter in a proximal position;

FIG. 1B depicts a perspective view of the dilation instrument assembly of FIG. 1A, with the guidewire in a distal position, and with the dilation catheter in the proximal position;

FIG. 1C depicts a perspective view of the dilation instrument assembly of FIG. 1A, with the guidewire in a distal position, with the dilation catheter in a distal position, and with a dilator of the dilation catheter in a non-dilated state;

FIG. 1D depicts a perspective view of the dilation instrument assembly of FIG. 1A, with the guidewire in a distal position, with the dilation catheter in the distal position, and with a dilator of the dilation catheter in a dilated state;

FIG. 2 depicts a schematic view of an exemplary sinus surgery navigation system being used on a patient seated in an exemplary medical procedure chair;

FIG. 3 depicts a side elevational view of an exemplary alternative navigation guidewire in communication with the navigation system of FIG. 2;

FIG. 4 depicts a cross-sectional view of a distal portion of the navigation guidewire of FIG. 3, the cross-section taken along line 4-4 of FIG. 3;

FIG. 5 depicts a side elevational view of another exemplary alternative navigation guidewire in communication with the navigation system of FIG. 2;

FIG. 6 depicts a cross-sectional view of a distal portion of the navigation guidewire of FIG. 5, the cross-section taken along line 6-6 of FIG. 5;

FIG. 7 depicts a perspective view of an exemplary illuminating guidewire in communication with a light source; and

FIG. 8 depicts a cross-sectional view of a distal portion of the illuminating guidewire of FIG. 7, the cross-section taken along line 8-8 of FIG. 7.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Dilation Catheter System

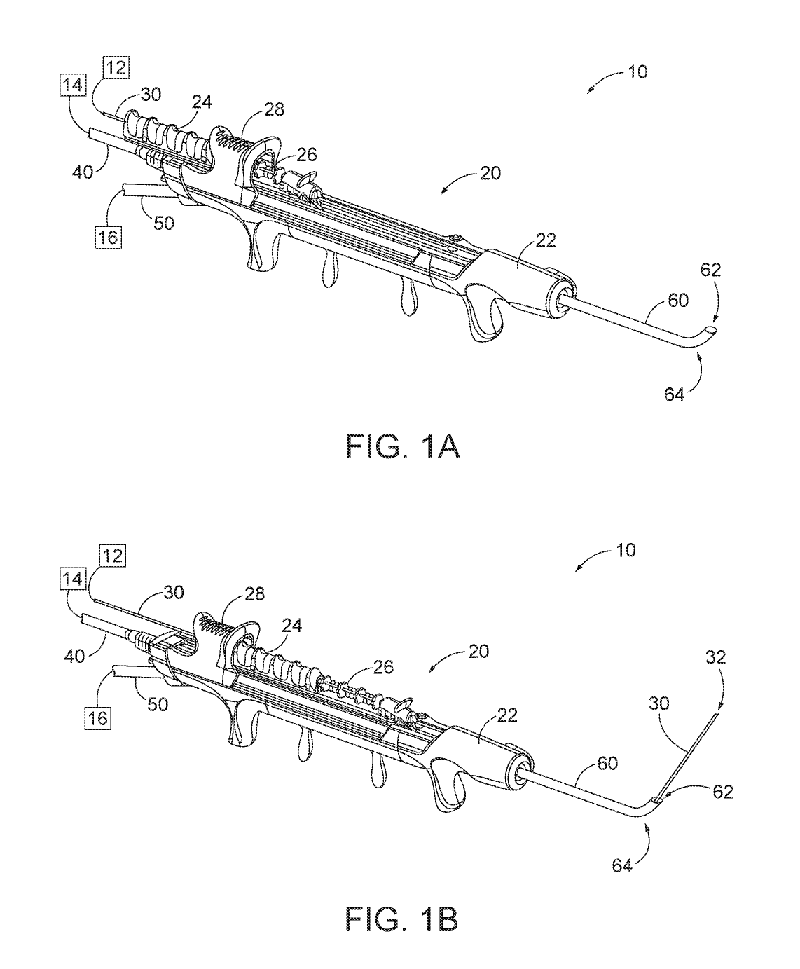

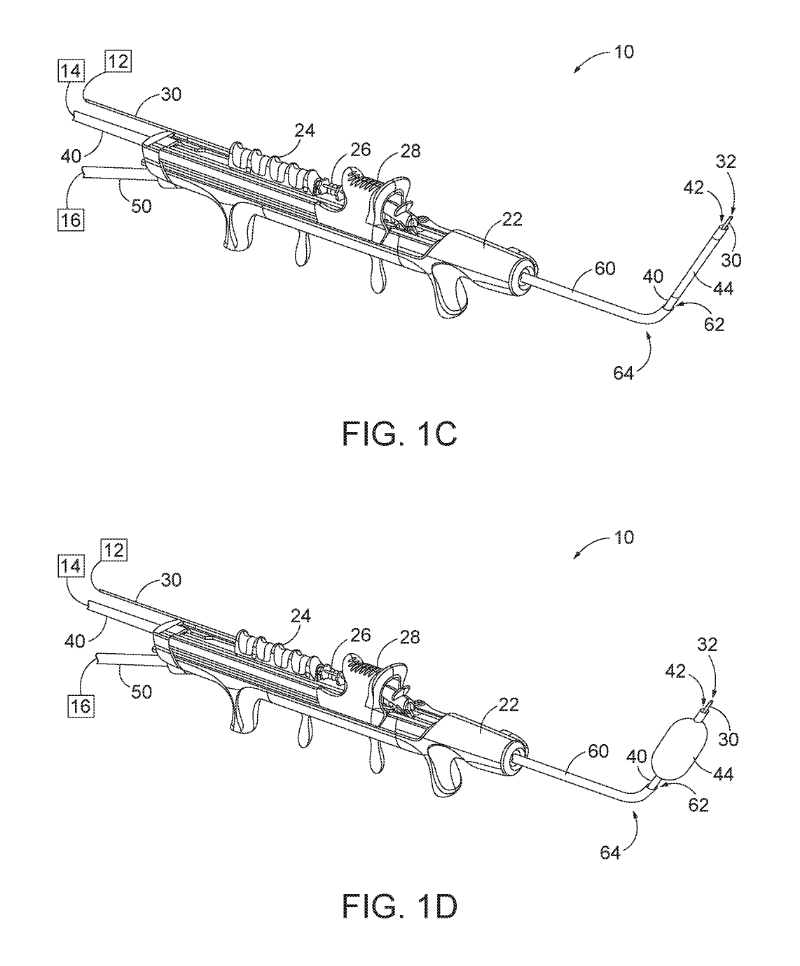

FIGS. 1A-ID show an exemplary dilation instrument assembly (10) that may be used to dilate the ostium of a paranasal sinus; to dilate some other passageway associated with drainage of a paranasal sinus; to dilate a Eustachian tube; or to dilate some other anatomical passageway (e.g., within the ear, nose, or throat, etc.). Dilation instrument assembly (10) of this example comprises a guidewire power source (12), an inflation source (14), an irrigation fluid source (16), and a dilation instrument (20). In some versions, guidewire power source (12) comprises a source of light. In some other versions, guidewire power source (12) is part of an IGS system as described below. Inflation source (14) may comprise a source of saline or any other suitable source of fluid. Irrigation fluid source (16) may comprise a source of saline or any other suitable source of fluid. Again, though, any other suitable source of fluid may be used. It should also be understood that irrigation fluid source (16) may be omitted in some versions.

Dilation instrument (20) of the present example comprise a handle body (22) with a guidewire slider (24), a guidewire spinner (26), and a dilation catheter slider (28). Handle body (22) is sized and configured to be gripped by a single hand of a human operator. Sliders (24, 28) and spinner (26) are also positioned and configured to be manipulated by the same hand that grasps handle body (22).

A guide catheter (60) extends distally from handle body (22). Guide catheter (60) includes an open distal end (62) and a bend (64) formed proximal to open distal end (62). Dilation instrument (20) is configured to removably receive several different kinds of guide catheters (60), each guide catheter (60) having a different angle formed by bend (64). Guide catheter (60) of the present example is formed of a rigid material (e.g., rigid metal and/or rigid plastic, etc.), such that guide catheter (60) maintains a consistent configuration of bend (64) during use of dilation instrument (20). In some versions, dilation instrument (20), is further configured to enable rotation of guide catheter (60), relative to handle body (22), about the longitudinal axis of the straight proximal portion of guide catheter (60), thereby further promoting access to various anatomical structures.

A guidewire (30) is coaxially disposed in guide catheter (60). Guidewire slider (24) is secured to guidewire (30). Translation of guidewire slider (24) relative to handle body (22) from a proximal position (FIG. 1A) to a distal position (FIG. 1B) causes corresponding translation of guidewire (30) relative to handle body (22) from a proximal position (FIG. 1A) to a distal position (FIG. 1B). When guidewire (30) is in a distal position, a distal portion of guidewire (30) protrudes distally from open distal end (62) of guide catheter (60). Guidewire spinner (26) is operable to rotate guidewire (30) about the longitudinal axis of guidewire (30). Guidewire spinner (26) is coupled with guidewire slider (24) such that guidewire spinner (26) translates longitudinally with guidewire slider (24). By way of example only, guidewire (30) may be configured in accordance with at least some of the teachings of U.S. Pat. No. 9,155,492, the disclosure of which is incorporated by reference herein. In some versions, dilation instrument (20) is configured to enable guidewire (30) to be completely removed from the rest of dilation instrument (20). Thus, guidewire (30) need not necessarily be permanently secured to other components of dilation instrument (20). Other features and operabilities that may be incorporated into guidewire (30) will be apparent to those of ordinary skill in the art in view of the teachings herein.

A dilation catheter (40) is coaxially disposed in guide catheter (60). Dilation catheter slider (28) is secured to dilation catheter (40). Translation of dilation catheter slider (28) relative to handle body (22) from a proximal position (FIG. 1B) to a distal position (FIG. 1C) causes corresponding translation of dilation catheter (40) relative to handle body (22) from a proximal position (FIG. 1B) to a distal position (FIG. 1C). When dilation catheter (40) is in a distal position, a distal portion of dilation catheter (40) protrudes distally from open distal end (62) of guide catheter (60). Dilation catheter (40) of the present example comprises a non-extensible balloon (44) located just proximal to open distal end (42) of dilation catheter (40). Balloon (44) is in fluid communication with inflation source (14). Inflation source (14) is configured to communicate fluid (e.g., saline, etc.) to and from balloon (44) to thereby transition balloon (44) between a non-inflated state and an inflated state. FIG. 1C shows balloon (44) in a non-inflated state. FIG. 1D shows balloon (44) in an inflated state. In the non-inflated state, balloon (44) is configured to be inserted into a constricted anatomical passageway. In the inflated state, balloon (44) is configured to dilate the anatomical passageway in which balloon (44) is inserted. Other features and operabilities that may be incorporated into dilation catheter (40) will be apparent to those of ordinary skill in the art in view of the teachings herein.

II. Exemplary Image Guided Surgery Navigation System

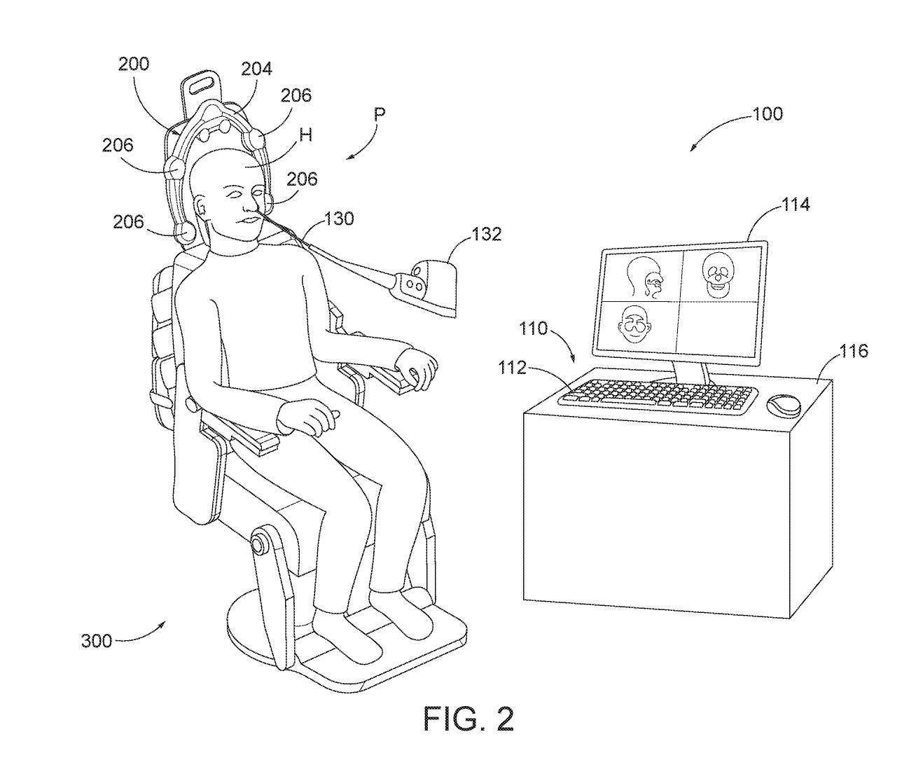

FIG. 2 shows an exemplary IGS navigation system (100) enabling an ENT procedure to be performed using image guidance. In some instances, IGS navigation system (100) is used during a procedure where dilation instrument assembly (10) is used to dilate the ostium of a paranasal sinus; or to dilate some other anatomical passageway (e.g., within the ear, nose, or throat, etc.). In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pub. No. 2016/0310042, entitled “System and Method to Map Structures of Nasal Cavity,” published Oct. 27, 2016; and U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein.

IGS navigation system (100) of the present example comprises a field generator assembly (200), which comprises set of magnetic field generators (206) that are integrated into a horseshoe-shaped frame (204). Field generators (206) are operable to generate alternating magnetic fields of different frequencies around the head of the patient. Field generators (206) thereby enable tracking of the position of a navigation guidewire (130) that is inserted into the head of the patient. Various suitable components that may be used to form and drive field generators (206) will be apparent to those of ordinary skill in the art in view of the teachings herein.

In the present example, frame (204) is mounted to a chair (300), with the patient (P) being seated in the chair (300) such that frame (204) is located adjacent to the head (H) of the patient (P). By way of example only, chair (300) and/or field generator assembly (200) may be configured and operable in accordance with at least some of the teachings of U.S. Patent App. No. 62/555,824, entitled “Apparatus to Secure Field Generating Device to Chair,” filed Sep. 8, 2017, the disclosure of which is incorporated by reference herein.

IGS navigation system (100) of the present example further comprises a processor (110), which controls field generators (206) and other elements of IGS navigation system (100). For instance, processor (110) is operable to drive field generators (206) to generate electromagnetic fields; and process signals from navigation guidewire (130) to determine the location of a sensor in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

A coupling unit (132) is secured to the proximal end of a navigation guidewire (130). Coupling unit (132) of this example is configured to provide wireless communication of data and other signals between console (116) and navigation guidewire (130). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Navigation guidewire (130) may be used as a substitute for guidewire (30) in dilation instrument (20) described above. Navigation guidewire (130) includes a sensor (not shown) that is responsive to movement within the fields generated by field generators (206). In the present example, the sensor of navigation guidewire (130) comprises at least one coil at the distal end of navigation guidewire (130). When such a coil is positioned within an electromagnetic field generated by field generators (206), movement of the coil within that magnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end of navigation guidewire (130) within a three-dimensional space (i.e., within the head (H) of the patient (P)). To accomplish this, processor (110) executes an algorithm to calculate location coordinates of the distal end of navigation guidewire (130) from the position related signals of the coil(s) in navigation guidewire (130).

Processor (110) uses software stored in a memory of processor (110) to calibrate and operate system (100). Such operation includes driving field generators (206), processing data from navigation guidewire (130), processing data from operating controls (112), and driving display screen (114). Processor (110) is further operable to provide video in real time via display screen (114), showing the position of the distal end of navigation guidewire (130) in relation to a video camera image of the patient's head (H), a CT scan image of the patient's head (H), and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. By way of example only, display screen (114) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (114).

The images provided through display screen (114) may help guide the operator in maneuvering and otherwise manipulating instruments within the patient's head. When used as a substitute for guidewire (30) in dilation instrument assembly (10), navigation guidewire (130) may facilitate navigation of instrumentation of dilation instrument assembly (10) within the patient during performance of a procedure to dilate the ostium of a paranasal sinus; or to dilate some other anatomical passageway (e.g., within the ear, nose, or throat, etc.). It should also be understood that other components of dilation instrument assembly (10) may incorporate a sensor like the sensor of navigation guidewire (130), including but not limited to dilation catheter (40).

III. Exemplary Alternative Reusable Guidewire

Some medical instruments that are commonly utilized in a medical may be commonly disposed of after a single use of the equipment during a medical procedure. This may result in cost and waste that might otherwise be reduced. In some instances, it may be desirable to utilize medical instruments that are capable of being reused in subsequent medical procedures to thereby reduce the overall costs of purchasing new instruments for each procedure. Employing medical instruments that may be reused in subsequent medical procedures may be further beneficial to minimize the overall waste produced after each completed medical procedure from the disposal of this used equipment. However, medical instruments that are intended to be reused may need to be sterilized after each procedure to effectively reduce the bioburden level of the instrument after its last use. Decreasing the bioburden level of some instruments may be particularly difficult due to the small diameters and sometimes irregular profiles of the medical instruments. In some cases, exposing a medical instrument to a disinfectant, detergent, or other aspect associated with cleaning and sterilization may not effectively achieve the desired level of bioburden reduction efficacy when the instrument comprises numerous gaps and crevices that are capable of housing various bioburdens and other particles. Moreover, some medical instruments may lack sufficient durability to withstand repeated cleaning, sterilization, and re-use.

Providing a guidewire, such as guidewires (30, 130) described above, that includes an elongated-tubular configuration, in lieu of a coiled-wire configuration, may be beneficial to permit for the reuse of guidewires (30, 130) for multiple procedures, thereby effectively extending the active lifecycle of the instrument. Having a guidewire that includes an outer tube that is does not include an irregular profile or narrow spaces between coil windings may be desirable to effectively achieve bioburden reduction of the guidewire. A tubular guidewire may also provide enhanced durability relative to a guidewire formed by a wound coil. Forming guidewire (30, 130) with an outer tubular configuration may allow the guidewire to be effectively sterilized and subsequently reused in multiple procedures with relative ease.

The following description provides various examples of a guidewire that is configured to be sterilized and reused in multiple medical procedures. It should be understood that the guidewires described below may be readily incorporated into any of the various dilation instrument assemblies (10) described above and in any of the various procedures described in the various references described herein. It should also be understood that the guidewires described below may be removed from the rest of the components of dilation instrument assembly (10), such that the guidewire may be subject to its own sterilization/reprocessing procedure. Other suitable ways in which the below-described guidewires may be used will be apparent to those of ordinary skill in the art in view of the teachings herein.

A. Exemplary Dual-Tube Navigation Guidewire

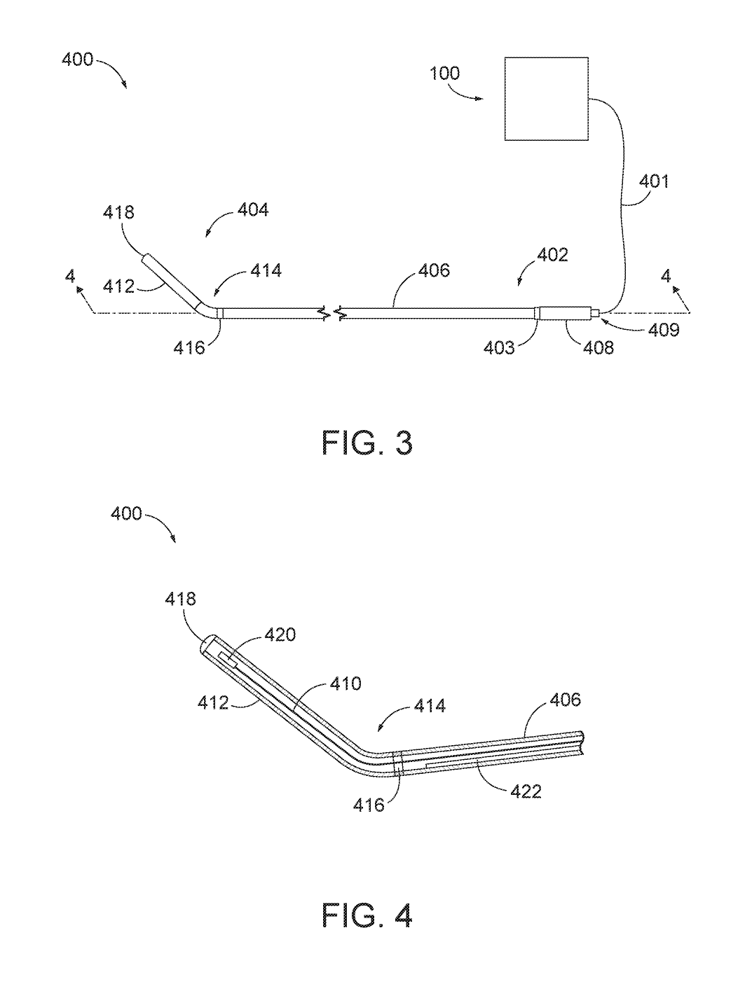

FIG. 3 shows an exemplary navigation guidewire (400) operatively connected to IGS navigation system (100) through a wired connection (401) such that navigation guidewire (400) is in communication with IGS navigation system (100). Alternatively, navigation guidewire (400) may be connected to IGS navigation system (100) through other suitable communication means (e.g., wireless communication, etc.). Except as otherwise described below, navigation guidewire (400) may be configured and operable just like navigation guidewire (130) described above. Accordingly, guidewire (400) of the present example may be readily incorporated into dilation instrument (20) of dilation instrument assembly (10) described above. A version of dilation instrument (20) that is equipped with navigation instrument (400) of the present example may be configured and operable similar to dilation instrument (20) described above, except for the differences discussed below.

Navigation guidewire (400) is operable to facilitate navigation of instrumentation of dilation instrument assembly (10) within a patient during performance of a procedure to dilate the ostium of a paranasal sinus; or to dilate some other anatomical passageway (e.g., within the ear, nose, or throat, etc.). Navigation guidewire (400) comprises a proximal end (402) and a distal end (404) separated by an elongate tube (406) and a bent hypotube (412). Elongate tube (406) and bent hypotube (412) each comprise a tubular shape such that an exterior surface of tubes (406, 412) is not coiled; nor does it otherwise have a profile comprising narrow gaps or cervices positioned along a longitudinal length of elongate tube (406), proximal end (402), and distal end (404), respectively. In other words, tube (406) and hypotube (412) each have a smooth outer surface along their entire respective lengths. Navigation guidewire (400) may have any suitable length as will be apparent to those of ordinary skill in the art.

Elongate tube (406) and proximal end (402) are each formed of a material suitable for exposure to a surgical instrument sterilizer. In other words, the exterior material of elongate tube (406) and end (402) are configured to withstand exposure to the various sterilizing fluids, vapors, plasma, etc. commonly encountered within instrument sterilizing machines that sterilize surgical instruments. Accordingly, elongate tube (406) and end (402) are formed of a durable material that is configured to encounter environmental conditions and processes that effectively sterilize navigation guidewire (400) while maintaining the structural integrity of navigation guidewire (400) for subsequent use in future medical procedures. By way of example only, elongate tube (406) and proximal end (502) may be formed of nitinol, polytetrafluoroethylene, metals with a high tolerance to chemical exposure, and/or various other suitable chemical-resistant materials as will be apparent to those of ordinary skill in the art.

Proximal end (402) includes a coupling unit (408) that is generally configured to operatively connect navigation guidewire (400) to IGS navigation system (100) such that coupling unit (408) provides communication of data and other signals between navigation guidewire (400) and console (116). In the present example, coupling unit (408) comprises a navigation 3-pin (409) connector that couples a sensor (420) (see FIG. 4) of navigation guidewire (400) to IGS navigation system (100). Coupling unit (408) encapsulates various electronics (not shown) suitable for transmitting power and memory from IGS navigation system (100) to sensor (420) through an electrical conduit (410). By way of example only, coupling unit (408) may include a memory and processor that are operable to read and transmit various processing values between navigation guidewire (400) and IGS navigation system (100). Coupling unit (408) includes a plurality of pins (409) extending proximally from coupling unit (408), with pins (409) being in communication with sensor (420) via electrical conduit (410). Coupling unit (408) of the present example is formed of stainless steel, though any other suitable material(s) may be used. Other suitable components and configurations that may be incorporated into coupling unit (408) will be apparent to those of ordinary skill in the art in view of the teachings herein.

Sensor (420) is disposed within distal end (404) and is operatively connected to an electrical conduit (410) that extends to proximal end (402). Sensor (420) is configured to be responsive to movement within the fields generated by field generators (206). In the present example, sensor (420) of navigation guidewire (400) comprises at least one coil at distal end (404) of navigation guidewire (400). When such a coil is positioned within an electromagnetic field generated by field generators (206), movement of the coil within that magnetic field may generate electrical current in the coil, and this electrical current may be communicated along electrical conduit (410) in navigation guidewire (400) and further to processor (110) via coupling unit (408). In response to the movement of distal end (404) and sensor (420) disposed therein, IGS navigation system (100) is operable to determine the location of distal end (404) of navigation guidewire (400) within a three-dimensional space (i.e., within the head (H) of the patient (P)). To accomplish this processor (110) executes an algorithm to calculate location coordinates of distal end (404) of navigation guidewire (400) from the position related signals of the coil(s) in navigation guidewire (400).

Proximal end (402) further includes a knob (403) positioned distal to coupling unit (408). Knob (403) is fixedly attached to elongate tube (406) and is configured to rotate such that knob (403) is operable to rotate elongate tube (406) and distal end (404) of navigation guidewire (400) relative to coupling unit (408). In the present example, knob (403) is solder welded to elongate tube (406) and is rotatably coupled to coupling unit (408) such that rotation of knob (403) relative to coupling unit (408) provides for the simultaneous rotation of elongate tube (406) and distal end (402) of navigation guidewire (400). In other versions, knob (403) may be laser welded to elongate tube (406) and/or securely fixed to elongate tube (406) via other suitable attachment means as will be apparent to those of ordinary skill in the art. In the present example, coupling unit (408), pins (409) and knob (403) are hermetically sealed such that the connections of coupling unit (408), pins (409) and knob (403) are fluid tight.

Bent hypotube (412) is sized and shaped to extend transversely from elongate tube (406) at a preformed angle (414). Although angle (414) is shown and described as being pre-formed, in other versions bent hypotube (412) may be malleable, such that hypotube (412) may provide a selectively adjustable bend angle (414). As best seen in FIG. 4, bent hypotube (412) is securely fastened to elongate tube (406) at a solder joint (416). In this instance, with bent hypotube (412) fixedly attached to elongate tube (406) at solder joint (416), bent hypotube (412) is operable to move unitarily with elongate tube (406) in response to rotation of elongate tube (406) by knob (403).

Opposite from solder joint (416), bent hypotube (412) includes an atraumatic tip (418) at a distal end of bent hypotube (412). Atraumatic tip (418) is sized, shaped, and configured to avoid causing tissue injury as navigation guidewire (400) is advanced into a patient during a medical procedure. By way of example only, atraumatic tip (418) may be formed of a solder material, glued glass, a cured adhesive material, and/or any other suitable material(s). In the present example, bent hypotube (412) is not formed of a similar material as elongate tube (406) and proximal end (402). By way of example only, hypotube (412) may be formed of stainless steel and/or any other suitable material(s).

Sensor (420) is disposed within bent hypotube (412) adjacent to atraumatic tip (418). Sensor (420) is configured to communicate with the electronics contained in coupling unit (408) by means of electrical conduit (410). Accordingly, electrical conduit (410) is connected to sensor (420) within bent hypotube (418) and extends through elongate tube (406) until terminating at its connection with coupling unit (408). As further seen in FIG. 4, navigation guidewire (400) further includes a core wire (422) extending through elongate tube (406) adjacent to electrical conduit (410). Core wire (422) is configured to provide additional column strength to elongate tube (406), thereby reducing the occurrence of buckling of elongate tube (406) when elongate tube (406) is pushed. The distal end of core wire (422) is soldered (or welded or otherwise fixedly secured) to a distal interior portion of elongate tube (406) proximal to solder joint (416); while the proximal end of core wire (422) is soldered (or welded or otherwise fixedly secured) to a proximal interior portion of elongate tube (406). As another variation, the distal end of core wire (422) may be soldered (or welded or otherwise fixedly secured) to bent hypotube (416) adjacent to solder joint (416), adjacent to atraumatic tip (418), and/or at various other suitable longitudinal positions within navigation guidewire (400). In some other versions, core wire (422) is simply omitted altogether.

In an exemplary use, navigation guidewire (400) is inserted into an anatomical passageway of a patient (e.g., within the ear, nose, or throat, etc.) after the head (H) of the patient (P) is located adjacent to frame (204). With field generators (206) generating magnetic fields of different frequencies around the head (H) of the patient (P), bent hypotube (412) is inserted into a targeted anatomical region within the head (H). Sensor (420) responds to the movement of bent hypotube (412) within the fields generated by field generators (206) such that IGS navigation system (100) and sensor (420) cooperatively enable tracking of distal end (404) of navigation guidewire (400) within the head (H) of the patient (P). With IGS navigation system (100) being used to verify proper positioning of navigation guidewire (400), dilation instrument (20) is manipulated to advance dilation catheter (40) along navigation guidewire (400) to thereby position balloon (44) in the targeted anatomical passageway. The balloon (44) is then inflated to dilate the targeted anatomical passageway.

At the conclusion of the medical procedure, navigation guidewire (400) is disassembled from dilation instrument (20) and inserted into a sterilization machine for subsequent sterilization and reprocessing. Due to the tubular configuration of bent hypotube (412) and elongate tube (406), the chemical fluids/vapors are able to effectively access and sterilize the substantial perimeter of navigation guidewire (400). Further, with elongate tube (406) and hypotube (412) being formed of a material configured to withstand exposure to the various environmental conditions provided within a sterilization machine, navigation guidewire (400) effectively maintains its structural durability despite being exposed to these environmental conditions that are necessary for achieving the requisite bioburden reduction. Once navigation guidewire (400) is effectively reprocessed, an operator may utilize navigation guidewire (400) in a subsequent medical procedure.

B. Exemplary Single-Tube Navigation Guidewire

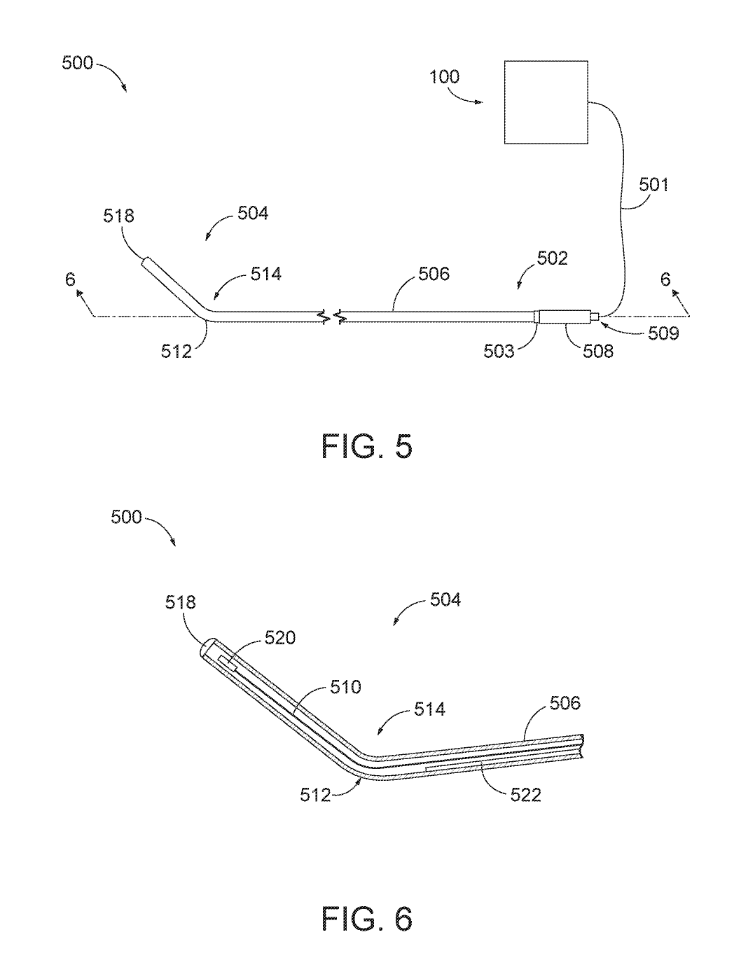

FIG. 5 shows an exemplary navigation guidewire (500) operatively connected to IGS navigation system (100) through a wired connection (501) such that navigation guidewire (500) is in communication with IGS navigation system (100). Alternatively, navigation guidewire (500) may be connected to IGS navigation system (100) through other suitable communication means (e.g., wireless communication, etc.). Except as otherwise described below, navigation guidewire (500) may be configured and operable just like navigation guidewire (130, 400) described above. Accordingly, guidewire (500) of the present example may be readily incorporated into dilation instrument (20) of dilation instrument assembly (10) described above. A version of dilation instrument (20) that is equipped with navigation instrument (500) of the present example may be configured and operable similar to dilation instrument (20) described above, except for the differences discussed below.

Navigation guidewire (500) is operable to facilitate navigation of instrumentation of dilation instrument assembly (10) within a patient during performance of a procedure to dilate the ostium of a paranasal sinus; or to dilate some other anatomical passageway (e.g., within the ear, nose, or throat, etc.). Navigation guidewire (500) comprises a proximal end (502) and a distal end (504) separated by an elongate tube (506). Elongate tube (506) comprise a tubular shape such that an exterior surface of elongate tube (506) is not coiled; nor does it otherwise have a profile comprising narrow gaps or cervices positioned along a longitudinal length of elongate tube (506). In other words, elongate tube (506) has a smooth outer surface along its entire length. Navigation guidewire (500) may have any suitable length as will be apparent to those of ordinary skill in the art.

Elongate tube (506), proximal end (502), and distal end (504) are formed of a material suitable for exposure to a surgical instrument sterilizer. In other words, the exterior material of elongate tube (506) and ends (502, 504) is configured to withstand exposure to the various sterilizing fluids, vapors, plasma, etc. commonly encountered within instrument sterilizing machines that sterilize surgical instruments. Accordingly, elongate tube (506) and ends (502, 504) are formed of a durable material that is configured to encounter environmental conditions and processes that effectively sterilize navigation guidewire (500) while maintaining the structural integrity of navigation guidewire (500) for subsequent use in future medical procedures. By way of example only, elongate tube (506) and ends (502, 504) may be formed of nitinol, polytetrafluoroethylene, metals with a high tolerance to chemical exposure, and/or various other suitable chemical-resistant materials as will be apparent to those of ordinary skill in the art.

Proximal end (502) includes a coupling unit (508) that is generally configured to operatively connect navigation guidewire (500) to IGS navigation system (100) such that coupling unit (508) provides communication of data and other signals between navigation guidewire (500) and console (116). Coupling unit (508) may be configured and operable just like coupling unit (408) of navigation guidewire (400) described above such that coupling unit (508) functions substantially similar to coupling unit (408). Proximal end (502) further includes a knob (503) positioned distal to coupling unit (508). Knob (503) is fixedly attached to elongate tube (506) and is configured to rotate such that knob (503) is operable to rotate elongate tube (506) and distal end (504) of navigation guidewire (500) relative to coupling unit (508). In other words, knob (503) is solder welded to elongate tube (506) and rotatably coupled to coupling unit (508) such that rotation of knob (503) relative to coupling unit (508) provides for the unitary rotation of elongate tube (506) and distal end (502) of navigation guidewire (500). In other versions, knob (503) may be laser welded to elongate tube (506) and/or securely fixed to elongate tube (506) via other suitable attachment means as will be apparent to those of ordinary skill in the art. In the present example, coupling unit (508), pins (509) and knob (503) are hermetically sealed such that the connections of coupling unit (508), pins (509) and knob (503) are fluid tight.

Distal end (504) includes a sensor (520) (see FIG. 6) operatively connected to an electrical conduit (510) that extends through elongate tube (506) and to proximal end (502). Coupling unit (508) electrically couples sensor (520) of navigation guidewire (500) to IGS navigation system (100). Sensor (520) is configured to be responsive to movement within the fields generated by field generators (206). In the present example, sensor (520) of navigation guidewire (500) comprises at least one coil at distal end (504) of navigation guidewire (500). Sensor (520) may be configured and operable just like sensor (420) of navigation guidewire (400) described above such that sensor (520) functions substantially similar to sensor (420).

Elongated tube (506) includes a bend (512) adjacent to distal end (504). Bend (512) is sized and shaped to extend distal end (504) transversely relative to proximal end (502) at a preformed angle (514). Although angle (514) is shown and described as being pre-formed, it should be understood that in other versions elongate tube (506) may provide malleability, such that elongate tube (506) may include a selectively adjustable bend angle (514), such that the orientation of bend (512) is not rigidly fixed relative to proximal end (502). In the present example, with distal end (504) being integrally formed with elongate tube (506) and proximal end (502), distal end (504) is operable to move unitarily with elongate tube (506) in response to rotation of proximal end (502) by knob (503).

Elongate tube (506) further includes an atraumatic tip (518) at distal end (504). Atraumatic tip (518) is configured and operable just like atraumatic tip (418) described above. Distal end (504) of navigation guidewire (500) further includes sensor (520) disposed distally relative to bend (512) and positioned adjacent to atraumatic tip (518). Sensor (520), similar to sensor (420), is configured to communicate with the electronics contained in coupling unit (508) via electrical conduit (510). Accordingly, electrical conduit (510) extends from sensor (520) and through elongate tube (506) until connecting with coupling unit (508) at proximal end (502).

As further seen in FIG. 6, navigation guidewire (500) further includes a core wire (522) extending through elongate tube (506) and positioned adjacent to electrical conduit (510). Core wire (522) is configured to provide additional column strength to elongate tube (506), thereby reducing the occurrence of buckling of elongate tube (506) when elongate tube (506) is pushed. The distal end of core wire (522) is soldered (or welded or otherwise fixedly secured) to a distal interior portion of elongate tube (506) proximal to bend (512), while the proximal end of core wire (522) is soldered (or welded or otherwise fixedly secured) to a proximal interior portion of elongate tube (506). As another variation, the distal end of core wire (522) may be soldered (or welded or otherwise fixedly secured) to elongate tube (506) distally relative to bend (512), adjacent to atraumatic tip (518), and/or at various other suitable longitudinal positions within navigation guidewire (500). It should also be understood that including core wire (522) disposed within elongate tube (506) is merely optional such that navigation guidewire (500) may entirely omit core wire (522).

In an exemplary use, navigation guidewire (500) is inserted into an anatomical passageway of a patient (e.g., within the ear, nose, or throat, etc.) after the head (H) of the patient (P) is located adjacent to frame (204). With field generators (206) generating magnetic fields of different frequencies around the head (H) of the patient (P), distal end (504) is inserted into a targeted anatomical region within the head (H). Sensor (520) responds to the movement of distal end (504) within the fields generated by field generators (206) such that IGS navigation system (100) and sensor (520) cooperatively enable tracking of distal end (504) of navigation guidewire (500) within the head (H) of the patient (P). With IGS navigation system (100) being used to verify proper positioning of navigation guidewire (500), dilation instrument (20) is manipulated to advance dilation catheter (40) along navigation guidewire (500) to thereby position balloon (44) in the targeted anatomical passageway. Balloon (44) is then inflated to dilate the targeted anatomical passageway.

At the conclusion of the ENT procedure, navigation guidewire (500) is disassembled from dilation instrument (20) and inserted into a sterilization machine for sterilization and reprocessing. Due to the tubular configuration of elongate tube (506), the chemical fluids/vapors are able to effectively access and sterilize the substantial perimeter of navigation guidewire (500). Further, with elongate tube (506) being formed of a material configured to withstand exposure to the various environmental conditions provided within the sterilization machine, navigation guidewire (500) effectively maintains its structural durability despite being exposed to these environmental conditions that are necessary for achieving the requisite bioburden reduction. Once navigation guidewire (500) is effectively reprocessed, an operator may utilize navigation guidewire (500) in a subsequent medical procedure.

C. Exemplary Tubular Illuminating Guidewire

While an endoscope may be used to provide visualization within an anatomical passageway, it may also be desirable to provide additional visual confirmation of the proper positioning of balloon (44) in the anatomical passageway before inflating balloon (44) in the anatomical passageway. This may be done using an illuminating version of guidewire (30). Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end of the illuminating guidewire is positioned in the maxillary sinus, the light may be visible through the patient's cheek. Using such external visualization to confirm the position of the illuminating guidewire, balloon (44) may then be advanced distally along the illuminating guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein.

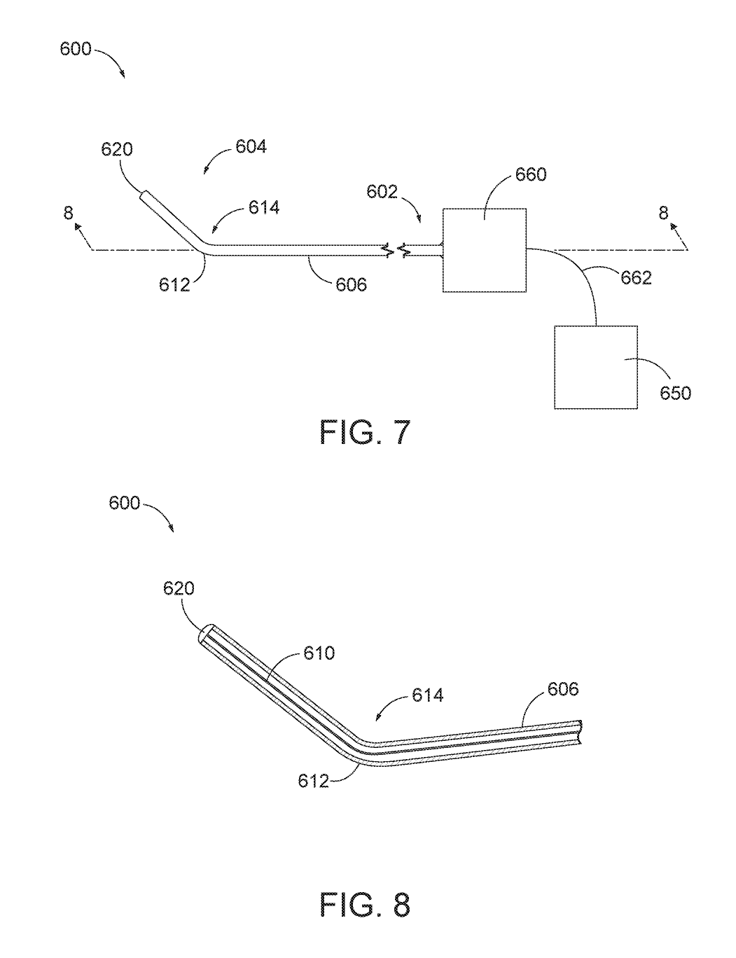

FIG. 7 shows an exemplary illuminating guidewire (600) optically coupled to a light source (650) through a coupling (660) and a light cable (662), such that light is communicated to illuminating guidewire (600) from light source (650). Except as otherwise described below, illuminating guidewire (600) may be configured and operable just like guidewire (30) described above. Accordingly, illuminating guidewire (600) of the present example may be readily incorporated into dilation instrument (20) of dilation instrument assembly (10) described above. A version of dilation instrument (20) that is equipped with illuminating guidewire (600) of the present example may be configured and operable similar to dilation instrument (20) described above, except for the differences discussed below.

Illuminating guidewire (600) is operable to provide illumination within a patient's anatomical passageway when used in conjunction with dilation instrument (20). Illuminating guidewire (600) comprises a proximal end (602) and a distal end (604) separated by an elongate tube (606). Elongate tube (606) comprise a tubular shape such that an exterior surface of elongate tube (606) is not coiled; nor does it otherwise have a profile comprising narrow gaps or cervices positioned along a longitudinal length of elongate tube (606). In other words, elongate tube (606) has a smooth outer surface along its entire length. Navigation guidewire (600) may have any suitable length as will be apparent to those of ordinary skill in the art.

Elongate tube (606), proximal end (602), and distal end (604) are formed of a material suitable for exposure to a surgical instrument sterilizer. In other words, the exterior material of elongate tube (606) and ends (602, 604) is configured to withstand exposure to the various sterilizing fluids, vapors, plasma, etc. commonly encountered within instrument sterilizing machines that sterilize surgical instruments. Accordingly, elongate tube (606) and ends (602, 604) are formed of a durable material that is configured to encounter environmental conditions and processes that effectively sterilize illuminating guidewire (600) while maintaining the structural integrity of illuminating guidewire (600) for subsequent use in future medical procedures. By way of example only, elongate tube (406) may be formed of nitinol, polytetrafluoroethylene, metals with a high tolerance to chemical exposure, and/or various other suitable chemical-resistant materials as will be apparent to those of ordinary skill in the art.

Coupling (660) is unitarily secured to proximal end (602) and is configured to communicate light from light cable (662) to an optical fiber (610) contained in elongate tube (406). While just one optical fiber (610) is shown, some variations may include more than one optical fiber (610). Light cable (662) is further coupled with light source (650), such that optical fiber (610) receives light generated by light source (650) via light cable (662) and coupling (660). Various suitable forms that light source (650), coupling (660), and light cable (662) may take will be apparent to those of ordinary skill in the art in view of the teachings herein. In some variations, light source (650) is integrated directly into proximal end (602), such that light cable (662) is omitted.

Optical fiber (610) terminates distally in atraumatic lens (620), which is secured to distal end (604) of elongate tube (606). Atraumatic lens (620) optically connected to a distal end of optical fiber (610) such that lens (620) is in optical communication with proximal end (602) through optical fiber (610). Atraumatic lens (620) is optically transmissive and is thereby configured to project light outwardly from distal end (604) when optical fiber (610) is illuminated by light source (650). In this instance, optical fiber (610) is configured to transmit light from light source (650) through elongate tube (606) and to atraumatic lens (620). Atraumatic lens (620) may comprise glass, plastic, a cured adhesive, or some other material that is at least partially optically transmissive. Atraumatic lens (620) is operable to receive light from optical fiber (610) and thereby project the light distally from distal end (604).

In some versions, coupling (660) includes a rotary knob (not shown) that is secured to the proximal end of elongate tube (606). Such a knob may allow an operator to rotate elongate tube (606) about the longitudinal axis of elongate tube (606), relative to the rest of coupling (660). The interface between elongate tube (606) and coupling (660), as well as the rest of guidewire (600) may be hermetically sealed.

Elongate tube (606) includes a bend (612) along the longitudinal length between distal end (604) and proximal end (602). In particular, bend (612) is adjacent to distal end (604) and is sized and shaped to extend atraumatic lens (620) transversely relative to proximal end (602) at a preformed angle (614). Although angle (614) of bend (612) is shown and described as being pre-formed, it should be understood that in other versions elongate tube (606) may be malleable, such that bend (612) may include a selectively adjustable angle (614) such that the orientation of bend (612) along elongate tube (606) is not rigidly fixed relative to the longitudinal length of elongate tube (606). In this instance, with distal end (604) fixedly attached to elongate tube (606), atraumatic lens (620) is operable to move unitarily with elongate tube (606) in response to rotation of elongate tube (606). Atraumatic lens (420) is sized, shaped, and configured to avoid causing tissue injury as illuminating guidewire (600) is advanced into a patient during a medical procedure.

While not shown, guidewire (600) may also include a core wire, like core wires (422, 522) described above. Alternatively, guidewire (600) may lack a core wire altogether.

In an exemplary use, illuminating guidewire (600) is inserted into an anatomical passageway of a patient (e.g., within the ear, nose, or throat, etc.) for dilation. With distal end (604) positioned within the target area, light source (650) is activated to thereby transmit light through light cable (662) and coupling (660); and further through optical fiber (610) toward distal end (604) such that the light projects distally from atraumatic lens (620) of illuminating guidewire (600). This light may illuminate the adjacent tissue (e.g., hypodermis, subdermis, etc.) and thus be visible to the naked eye from outside the patient through transcutaneous illumination. For instance, when distal end (604) is positioned in the maxillary sinus, the light may be visible through the patient's cheek. Using such external visualization to confirm the position of illuminating guidewire (600), balloon (44) of dilation instrument (20) may then be advanced distally along illuminating guidewire (600) into position at the dilation site. Balloon (44) is then inflated to dilate the targeted anatomical passageway.

At the conclusion of the ENT procedure, illuminating guidewire (600) is disassembled from dilation instrument (20) and inserted into a sterilization machine for sterilization and reprocessing. Due to the tubular configuration of elongate tube (606), the chemical fluids/vapors are able to effectively access and sterilize the substantial perimeter of navigation guidewire (600). Further, with elongate tube (606) being formed of a material configured to withstand exposure to the various environmental conditions provided within the sterilization machine, navigation guidewire (600) effectively maintains its structural durability despite being exposed to these environmental conditions that are necessary for achieving the requisite bioburden reduction. Once navigation guidewire (500) is effectively reprocessed, an operator may utilize navigation guidewire (600) in a subsequent medical procedure.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A guidewire, comprising: (a) an elongate tube including: (i) a proximal portion, and (ii) distal portion, wherein the elongate tube has a tubular profile with a substantially smooth surface extending between the proximal portion and the distal portion, wherein the distal portion has an outer diameter sized to fit within a drainage passageway of a paranasal sinus, wherein the elongate tube is formed of a material configured to tolerate sterilization; (b) a connector coupled to the proximal portion of the elongate tube, wherein the connector is configured to communicate with a navigation system, wherein the elongate tube is rotatable relative to the connector; (c) a sensor positioned in or adjacent to the distal portion of the elongate tube; and (d) a conduit disposed within the elongate tube, wherein the conduit extends from the connector to the sensor, wherein the conduit is configured to communicate a signal from the sensor to the connector.

### Example 2

The guidewire of Example 1, wherein the elongate tube includes a bend along the distal portion such that the distal portion is transversely oriented relative to the proximal portion.

### Example 3

The guidewire of Example 2, wherein the distal portion comprises an atraumatic distal tip.

### Example 4

The guidewire of Example 3, wherein the atraumatic tip is formed of a solder material, metal, or glued glass.

### Example 5

The guidewire of Example 4, wherein the sensor is disposed within the distal portion adjacent to the atraumatic tip such that the sensor is configured to transmit a signal indicative of the location of the atraumatic tip to the navigation system.

### Example 6

The guidewire of any one or more of Examples 1 through 5, further comprising a hypotube securely attached to the distal portion of the elongate tube at a joint.

### Example 7

The guidewire of Example 6, wherein the joint comprises solder joining the hypotube to the distal portion of the elongate tube at the joint.

### Example 8

The guidewire of Example 6, wherein the joint comprises a weld joining the hypotube to the distal portion of the elongate tube at the joint.

### Example 9

The guidewire of any one or more of Examples 6 through 8, wherein the hypotube comprises a bend located distal to the joint such that the hypotube has a longitudinal length that is transverse to the elongate tube.

### Example 10

The guidewire of any one or more of Examples 6 through 9, wherein the hypotube is formed of stainless steel.

### Example 11

The guidewire of any one or more of Examples 6 through 10, wherein the sensor is disposed within the hypotube such that the conduit extends through the joint and into the hypotube.

### Example 12

The guidewire of any one or more of Examples 6 through 11, wherein the hypotube includes an atraumatic tip at an end of the hypotube opposite the joint.

### Example 13

The guidewire of any one or more of Examples 1 through 12, further comprising a dilation catheter, wherein the dilation catheter comprises: (i) a shaft defining a lumen, wherein the elongate tube is slidably disposed in the lumen, and (ii) an expandable dilator positioned at a distal portion of the shaft, wherein the expandable dilator in a non-expanded state is configured to fit within a drainage passageway of a paranasal sinus, wherein the expandable dilator in an expanded state is configured to dilate a drainage passageway of a paranasal sinus.

### Example 14

The guidewire of Example 13, further comprising a guide, wherein the dilation catheter is slidably disposed in the guide.

### Example 15

The guidewire of any one or more of Examples 1 through 14, wherein the elongate tube comprises nitinol.

### Example 16

The guidewire of any one or more of Examples 1 through 15, further comprising a core wire disposed within the elongate tube and extending from the proximal portion to a point proximate to the distal portion.

### Example 17

A guidewire, comprising: (a) an elongate tube having a longitudinal length extending between a proximal end and a distal end, wherein the longitudinal length has a substantially cylindrical shape, wherein the elongate tube is formed of a material configured to tolerate sterilization, wherein the distal end has an outer diameter sized to fit within a drainage passageway of a paranasal sinus; (b) a connector coupled to the proximal end of the elongate tube, wherein the connector is configured to communicate with a light source; and (c) a light transmitting fiber disposed within the elongate tube, wherein the light transmitting fiber is in optical communication with the connector such that the light transmitting fiber is configured to transmit light from the light source; (d) a lens positioned adjacent to the distal end of the elongate tube, wherein the lens is in optical communication with the light transmitting fiber such that the lens is configured to project light communicated from the light source via the light transmitting fiber.

### Example 18

A method of dilating an anatomical passageway of a patient, the method comprising: (a) inserting a distal portion of a guidewire into an anatomical passageway located within a head of a patient, wherein the guidewire comprises a tubular body defining a smooth outer surface, wherein the tubular body extends longitudinally to the distal portion of the guidewire, such that a distal portion of the tubular body is disposed in the anatomical passageway; (b) advancing a dilation catheter along the guidewire to position a dilator of the dilation catheter in the anatomical passageway; and (c) expanding the dilator to thereby dilate the anatomical passageway.

### Example 19

The method of Example 18, wherein the guidewire further includes a sensor located in the distal portion, the method further comprising operating a navigation system to observe real-time position information based on data from the sensor to confirm positioning of the distal portion of the tubular body in the anatomical passageway.

### Example 20

The method of Example 18, wherein the guidewire is configured to emit light through a distal end of the guidewire, the method further comprising observing transillumination from the emitted light via the face of the patient to confirm positioning of the distal portion of the tubular body in the anatomical passageway

V. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A guidewire, comprising:
(a) an elongate tube including:
(i) a proximal portion, and
(ii) a distal portion, wherein the elongate tube has a tubular profile with a substantially smooth surface extending between the proximal portion and the distal portion, wherein the distal portion has an outer diameter sized to fit within a drainage passageway of a paranasal sinus,
wherein the elongate tube is formed of a material configured to tolerate sterilization;
(b) a connector coupled to the proximal portion of the elongate tube, wherein the connector is configured to communicate with a navigation system, wherein the elongate tube is rotatable relative to the connector;
(c) a sensor positioned in or adjacent to the distal portion of the elongate tube; and
(d) a conduit disposed within the elongate tube, wherein the conduit extends from the connector to the sensor, wherein the conduit is configured to communicate a signal from the sensor to the connector.

2. The guidewire of claim 1, wherein the elongate tube includes a bend along the distal portion such that the distal portion is transversely oriented relative to the proximal portion.

3. The guidewire of claim 2, wherein the distal portion comprises an atraumatic distal tip.

4. The guidewire of claim 3, wherein the atraumatic tip is formed of a solder material, metal, or glued glass.

5. The guidewire of claim 4, wherein the sensor is disposed within the distal portion adjacent to the atraumatic tip such that the sensor is configured to transmit a signal indicative of the location of the atraumatic tip to the navigation system.

6. The guidewire of claim 1, further comprising a hypotube securely attached to the distal portion of the elongate tube at a joint.

7. The guidewire of claim 6, wherein the joint comprises solder joining the hypotube to the distal portion of the elongate tube at the joint.

8. The guidewire of claim 6, wherein the joint comprises a weld joining the hypotube to the distal portion of the elongate tube at the joint.

9. The guidewire of claim 6, wherein the hypotube comprises a bend located distal to the joint such that the hypotube has a longitudinal length that is transverse to the elongate tube.

10. The guidewire of claim 6, wherein the hypotube is formed of stainless steel.

11. The guidewire of claim 6, wherein the sensor is disposed within the hypotube such that the conduit extends through the joint and into the hypotube.

12. The guidewire of claim 6, wherein the hypotube includes an atraumatic tip at an end of the hypotube opposite the joint.

13. The guidewire of claim 1, further comprising a dilation catheter, wherein the dilation catheter comprises:
(i) a shaft defining a lumen, wherein the elongate tube is slidably disposed in the lumen, and
(ii) an expandable dilator positioned at a distal portion of the shaft, wherein the expandable dilator in a non-expanded state is configured to fit within a drainage passageway of a paranasal sinus, wherein the expandable dilator in an expanded state is configured to dilate a drainage passageway of a paranasal sinus.

14. The guidewire of claim 13, further comprising a guide, wherein the dilation catheter is slidably disposed in the guide.

15. The guidewire of claim 1, wherein the elongate tube comprises nitinol.

16. The guidewire of claim 1, further comprising a core wire disposed within the elongate tube and extending from the proximal portion to a point proximate to the distal portion.

17. A guidewire, comprising:
(a) an elongate tube having a longitudinal length extending between a proximal end and a distal end, wherein the longitudinal length has a substantially cylindrical shape, wherein the elongate tube is formed of a material configured to tolerate sterilization, wherein the distal end has an outer diameter sized to fit within a drainage passageway of a paranasal sinus;
(b) a connector coupled to the proximal end of the elongate tube, wherein the connector is configured to communicate with a light source; and
(c) a light transmitting fiber disposed within the elongate tube, wherein the light transmitting fiber is in optical communication with the connector such that the light transmitting fiber is configured to transmit light from the light source;
(d) a lens positioned adjacent to the distal end of the elongate tube, wherein the lens is in optical communication with the light transmitting fiber such that the lens is configured to project light communicated from the light source via the light transmitting fiber.

18. A method of dilating an anatomical passageway of a patient, the method comprising:
(a) inserting a distal portion of a guidewire into an anatomical passageway located within a head of a patient, wherein the guidewire comprises a tubular body defining a smooth outer surface, wherein the tubular body extends longitudinally to the distal portion of the guidewire, such that a distal portion of the tubular body is disposed in the anatomical passageway;
(b) advancing a dilation catheter along the guidewire to position a dilator of the dilation catheter in the anatomical passageway; and
(c) expanding the dilator to thereby dilate the anatomical passageway.

19. The method of claim 18, wherein the guidewire further includes a sensor located in the distal portion, the method further comprising operating a navigation system to observe real-time position information based on data from the sensor to confirm positioning of the distal portion of the tubular body in the anatomical passageway.

20. The method of claim 18, wherein the guidewire is configured to emit light through a distal end of the guidewire, the method further comprising observing transillumination from the emitted light via the face of the patient to confirm positioning of the distal portion of the tubular body in the anatomical passageway.
